(19) United States
(12) Patent Application Publication
(10) Pub. No.: US 2011/0190535 A1
(43) Pub. Date: Aug. 4, 2011

(54) PROCESS FOR PREPARING POLYAROMATIC POLYISOCYANATE COMPOSITIONS

(75) Inventors: Robert Henry Carr, Bertem (BE); Rabah Mouazer, Wavre (BE); Willem Van Der Borden, Vlaardingen (NL)

(73) Assignee: Huntsman International LLC, The Woodlands, TX (US)

(21) Appl. No.: 12/673,076

(22) PCT Filed: Jul. 11, 2008
(86) PCT No.: PCT/EP2008/059079

§ 371 Date: Apr. 22, 2010

(30) Foreign Application Priority Data
Aug. 23, 2007 (EP) 07114803.5

(51) Int. Cl. C07C 249/00; C07C 211/50

(52) U.S. Cl. 560/358; 564/315

(57) ABSTRACT

Process for the preparation of polyaromatic polyamines comprising the step of reacting formaldehyde with at least one monoaromatic monoamine and at least one monoaromatic compound containing at least two amino functions in the presence of an acidic catalyst where a) the total amount of di-aromatic compounds in the polyaromatic polyamine mixture is in the range from about 25 wt % to about 50 wt % and b) the amount of monoaromatic compound containing at least two amine functions is in the range 5 to 30 mole % relative to 100 mole % of the total amount of monoaromatic monoamines and c) the amount of acidic catalyst used in the preparation of the polyaromatic polyamine mixture is less than about 0.4 moles per mole of formaldehyde or formaldehyde equivalents.

### TECHNICAL FIELD

The present invention relates to polyaromatic polyisocyanate compositions especially beneficial for the manufacture of polyurethanes, polyisocyanurates, polyureas and related products by means of phosgenation of particular polyamine compositions produced by economically beneficial means.

### INTRODUCTION

Polyurethanes are a diverse group of polymeric materials with a huge global market. Despite extensive research by many commercial and academic organisations, only two aromatic polyisocyanates—toluene diisocyanate (TDI) and the poly-aromatic poly-isocyanate compositions known as polymeric MDI (hereafter PMDI)—have found widespread commercial use (see, for example, Chemistry and Technology of Isocyanates, H. Ulrich, John Wiley & Sons, 2001). The term PMDI as used herein is taken also to include various di-isocyanate compositions well known as MDI. Relatively minor amounts of other aromatic polyisocyanates such as para-phenylene di-isocyanate (PPDI) and naphthalene di-isocyanate (NDI) are also produced for specific applications. Other di-isocyanates produced commercially which contain aromatic groups are generally classed as aliphatics because the isocyanate group is not directly bonded to the aromatic ring e.g. xylene di-isocyanate (XDI) (see Ulrich, as above). The largest segments of the polyurethanes market are based on PMDI. Polyureas based on PMDI plus amine-functionalised materials are also well known. Other significant markets exist where PMDI is used directly, for example, as a binder for wood-based products, etc. Many diverse PMDI compositions are known, based on phosgenation and work-up of the corresponding polyamine mixtures, optionally followed by removal of some of the lower molecular weight di-isocyanate MDI compounds and also optionally with further changes for example by blending together of different di-isocyanate MDI's and PMDI's.

Despite the widespread applicability of the various PMDI compositions, it has been found to be advantageous in certain cases to use mixtures of isocyanates in order to obtain particular benefits. Such blends include those using di-isocyanates with higher NCO values and lower viscosities than PMDI compositions; for example U.S. Pat. No. 3,492,251 and U.S. Pat. No. 3,936,483 disclose TDI-PMDI mixtures. A serious disadvantage of such mixtures however is the presence of the TDI which, being significantly more volatile than MDI, requires more stringent requirements for polyurethane production workplace environments in order to protect personnel from any deleterious effects of the TDI vapors.

This therefore also impacts negatively on the economics of such production facilities. This disadvantage is likewise also present for mixtures which might be produced from PMDI with other relatively low molecular weight aliphatic or aromatic polyisocyanates such as isophorone di-isocyanate (IPDI) or PPDI.

In order to improve the properties of existing products based on polyaromatic polyisocyanates or to develop new applications or uses for such polyaromatic polyisocyanates, there remains a need for different polyaromatic polyisocyanate compositions with different combinations of important characteristics [isocyanate content (determined as NCO value), reactivity, viscosity and the like] but without the limitations associated with TDI/PMDI mixtures or other mononuclear aromatic polyisocyanate/PMDI mixtures. The new combinations of characteristics should however be broadly in line with existing PMDI products in order to minimise change-over costs for processing equipment (avoiding new equipment designs, new installations and the like) and which also avoid the use of relatively volatile constituents. A key parameter of existing commercial PMDI compositions which closely reflects the most important characteristics of the entire polymeric mixture is the content of diaromatic molecules. In such mixtures the content of diaromatic molecules is almost invariably greater than 25 wt % but less than about 50 wt % of the total polyaromatic polyisocyanate mixture. Creation of the desired different polyaromatic polyisocyanate compositions with different combinations of important characteristics can be achieved by introduction of different monomers into the corresponding conventional polyaromatic polyamine mixture. However, the additional monomer should be a relatively minor component in order to avoid the creation of a totally different polymeric mixture with resulting characteristics and properties which are likewise totally removed from those of the conventional PMDI products. As well as meeting specific compositional requirements, such polyaromatic polyisocyanate compositions must at the same time also be manufactured by economically viable processes.

Surprisingly, we have found that such a challenging objective can be met by producing polyaromatic polyisocyanates by phosgenation and work-up of polyaromatic polyamines based on reaction of formaldehyde with at least one monoaromatic monoamine, preferably aniline, and at least one monoaromatic diamine, preferably one or more of the toluene diamine isomers or one or more isomers of diaminobenzene or their mixtures, where the total content of diaromatic [so-called “di-nuclear”] molecules in the final polyaromatic polyamine mixture is greater than 25 wt % but less than about 50 wt % of the total polyaromatic polyamine and where the monoaromatic diamine is used in an amount between about 5 and about 30 mole % preferably between about 10 and about 25 mole % relative to 100 mole % of the monoaromatic monoamine used for the production and where the manufacturing processes are carried out by economically beneficial means especially for the polyamine stage by using relatively low levels of acid catalyst and without operating at economically unattractive slow rates. An additional benefit of the specific compositions is the creation of products to facilitate manufacture of polyurethane, polyisocyanurate and polyurea materials or other products with associated improved properties.

### PRIOR ART

A mixture of polyaromatic polyisocyanates different from PMDI, especially distinguished because of the different positional isomers produced by the chemical reactions, can be made by coupling of various mononuclear aromatic compounds together by means of Friedal-Crafts reactions or other suitable chemistry, followed by nitration to form nitro groups, subsequent hydrogenation to form amino groups and subsequent phosgenation to produce isocyanate groups [U.S. Pat. No. 4,613,687]. These polyisocyanates may also be reduced or partially reduced to form related products [U.S. Pat. No. 4,603,189, U.S. Pat. No. 4,675,437] whilst the polyamines may also be used without phosgenation [DE 3414803, DE 3414804]. Manufacture of such polyisocyanate products is economically unattractive compared to coupling of mononuclear monoamines and diamines followed by phosgenation because of several reasons including the additional production steps required, the additional chemical reagents required and the inability to carry out the production stages in predominantly the same production facilities as used to make conventional PMDI products.

The species consisting of one aniline monomer and one toluene diamine (TDA) monomer linked by a methylene group derived from formaldehyde when phosgenated gives a species which may be termed methylene di-phenylene, mono-methyl, tri-isocyanate [MTI] or triisocyanato-methyl-diphenylmethane. One MTI isomer—specifically 2,4,4′-triisocyanato-5-methyl-diphenylmethane—is a known compound [CAS: 24373-99-7] which can be used for making isocyanurates [GB 809809] and biurets [GB 1030305] and has also been used in studies measuring the functionality of polyisocyanates [Effective Functionality and Intramolecular Reactions of Polyisocyanates and Polyols, Macromolecules, 2 (No. 6), 581-587 (1969). It can also be mixed with other isocyanate products to make usable mixtures [JP-A-02178261]. Manufacture of such polyisocyanate products is economically unattractive compared to coupling of mononuclear monoamines and diamines followed by phosgenation because of several reasons including the additional production steps required to make the MTI and separately either TDI, MDI or PMDI for blending.

The present invention provides polyaromatic polyisocyanates by phosgenation and work-up of polyaromatic polyamines based on reaction of formaldehyde with at least one monoaromatic monoamine, preferably aniline, and at least one monoaromatic diamine, preferably one or more of the toluene diamine isomers or one or more isomers of diaminobenzene or their mixtures, where the total content of diaromatic [so-called “di-nuclear”] molecules in the final polyaromatic polyamine mixture is greater than 25 wt % but less than about 50 wt % of the total polyaromatic polyamine and where the monoaromatic diamine is used in an amount between about 5 and about 30 mole %, preferably between about 10 and about 25 mole %, relative to 100 mole % of the monoaromatic monoamine used for the production and where the polyamine manufacturing process is carried out by economically beneficial means especially by using relatively low levels of acid catalyst and without operating at economically unattractive slow rates.

The advantage of preparing such polyaromatic polyisocyanates without greatly increasing the production costs of the process should be viewed in the light of the fact that modern industrial PMDI plants are highly complicated, frequently operating continuously and have to meet very high requirements with regard to safety, “on-line” availability and reliability. It is therefore a further object of the present invention to provide a process which can be carried out with little additional technical complexity even in existing industrial PMDI production plants. The term “PMDI plant” is taken here to include all plant required for the manufacture and work-up of both the polyamine and polyisocyanate products and associated plant, whatever the geographical location and physical arrangements for example whether located on a single production site or separated.

Polyaromatic polyamines can be produced by condensation of formaldehyde with a wide range of starting amines or mixtures thereof [see, for example, U.S. Pat. No. 3,931,320 and U.S. Pat. No. 4,189,443] and, indeed, use of aniline and TDA has been described previously.

DD 254387 includes TDA as one component in a complex reaction system for undertaking a process to condense formaldehyde with aromatic amines using base catalysed chemistry. The disclosed method is very limited and does not address the targets of the present invention.

Additional disclosures on the preparations of polyaromatic polyamines using acid catalysts have been made.

U.S. Pat. No. 3,012,008 describes producing polyamine mixtures and, subsequently, the corresponding polyisocyanates by condensation of formaldehyde with mixtures of mononuclear aromatic amines such as aniline, toluidine, phenylene diamine, toluene diamine, etc. [various isomers are specified] in order to obtain dinuclear polyisocyanates. However, the proportion of total amines to be reacted with formaldehyde is large [ratio of total amines to formaldehyde equivalents equal to at least 4] in order to minimise the production of polynuclear polyamines. Such an approach to the production of polyamines is limited in its scope to the formation of predominantly dinuclear polyamines. Although not specifically described, the process is exemplified entirely using aqueous hydrochloric acid in about equimolar amount to the formaldehyde. Thus, the invention is also unattractive on an economical basis because the relatively large amount of acid catalyst employed requires neutralisation with an equivalent or excess quantity of base and the resulting salt effluent must be treated and disposed of at significant cost. Likewise, although not specifically described, the process is exemplified entirely using a formalin addition temperature below about 5° C. Thus, the invention is also unattractive on an economical basis because of the large amount of cooling energy and associated equipment required to operate on an industrial scale at such low temperature.

FR 2337709 describes a general method for the production of predominantly asymmetrical polyamines characterised by initial condensation of an aromatic primary amine with formaldehyde in the presence of an acid catalyst, followed by addition of a second more reactive aromatic primary amine. Preferably, acid catalyst is used at 0.1 to 1 mole per mole of the total of the starting amines used and it proved particularly advantageous to add the total quantity of acid catalyst used during the first reaction. 2,4′-TDA is listed as one of many possible amines to use. Although not explicitly described, the disclosed approach to the production of polyamines is limited in its scope to the formation of predominantly dinuclear polyamines.

JP 10001461 describes a process for making predominantly triamino-diphenylmethanes by condensing formaldehyde with acidic aniline at low temperature and then adding the appropriate aromatic diamine within a temperature range of 0 to 40° C., preferably in the range 20-30° C. in order to generate selectively the asymmetric triaminodiphenylmethanes without simultaneously forming the oligomeric forms. Likewise, the rearrangement reactions between the initially formed aminobenzylanilines and the added aromatic diamine are also predominantly carried out at relatively low temperature, preferably for an extended period at a temperature in the range 30 to 60° C. A higher temperature can be used to finish off the reaction. The polyamines may be used to manufacture the corresponding polyisocyanates or as initiators for polyether polyols [JP 11029635]. The disclosed approach to the production of polyamines is limited in its scope to the formation of predominantly dinuclear polyamines.

U.S. Pat. No. 4,162,358 describes preparation of a polyamine mixture for use as an epoxy curing agent by reaction of formaldehyde with aromatic mono- and di-amines over a solid acid catalyst. Significant research has been undertaken over an extensive time period by a wide range of organisations into the use of a wide range of solid acid catalysts for manufacturing polyamine mixtures (e.g. Trends in industrial catalysis in the polyurethane industry, Wegener et al., Applied Catalysis A: General, (2001) 221, 303-335) but the disadvantages of the heterogeneous catalyst approach have thus far precluded significant use for commercial operation at the industrial scale.

U.S. Pat. No. 3,857,890 describes the production of polyfunctional polymethylene polyphenyl polyamine mixtures by first forming di(aminophenyl)methanes containing a high proportion of o,p′-isomer by reaction of aniline and formaldehyde in the presence of mineral acid, neutralising the intermediate mixture of aminobenzylanilines and, optionally, removing excess aniline and, subsequently, heating the aminobenzylanilines in the presence of an aromatic primary amine. The aromatic amine added in the final step could be 2,4-TDA. A key aspect of the invention is to maintain the temperature of the reaction mixture at a level low enough to minimise further conversion of the intermediate aminobenzylanilines to primary amines. However, neutralisation of the acid catalyst is an exothermic process which, thus, would apparently require additional process issues to be addressed. For example, the neutralisation step could be carried out especially slowly and/or additional cooling capacity could be included with the process equipment. Use of very low levels of acid catalyst, such that the neutralisation exotherm becomes insignificant to the reaction chemistry, implies significantly longer reaction times. In addition, we have found by means of our own research that neutralisation of the viscous organic mixture containing significant levels of aminobenzylanilines is more problematic, particularly at industrial scale, than neutralisation of the lower viscosity mixture of the corresponding primary amines present after the rearrangement reactions. Thus, the novelty of the described process brings significant additional detrimental issues both in terms of process complexity and economic performance.

U.S. Pat. No. 3,459,781 describes producing low viscosity and low volatility polyisocyanates from the corresponding polyamines by first condensing formaldehyde, in the presence of an acid catalyst, with at least about an equimolar amount of a monoamine, such as aniline, followed by addition of an aromatic diamine to the reaction mixture. In this way, the secondary amine intermediates formed in the initial monoamine-formaldehyde condensation react with the added diamine resulting in formation of the desired mixed amine products. Both the addition of formaldehyde to the monoamine and subsequent addition of diamine to the condensation reaction mixture are carried out at temperatures less than 50° C. According to the inventors, the exact amount of hydrochloric acid catalyst to be used is not critical although a practical range of 0.4 to 2 moles of acid per equivalent of amine is mentioned. However, even the lower level of acid disclosed requires neutralisation with at least the molar equivalent of a base such as sodium hydroxide which is economically undesirable compared to use of the significantly lower amounts which are employed in the present invention.

Thus, none of the prior art addresses the issues of large scale, economically beneficial production of the specific polyaromatic polyamine compositions of the present invention.

Thus there remains a need for different polyaromatic polyisocyanate compositions with different combinations of important characteristics [reactivity, viscosity and the like] to be manufactured from the corresponding polyaromatic polyamines by economically viable processes but without the limitations associated with TDI/PMDI or other mononuclear aromatic polyisocyanate/PMDI mixtures and which avoid the drawbacks of the prior art.

We have found that this object is achieved by the production of polyamine mixtures from formaldehyde, a mono-nuclear aromatic monoamine and one or more aromatic di- or higher functionality amines according to the present invention.

The present invention provides a process for the production of polyaromatic polyamines where the total content of diaromatic [so-called “di-nuclear”] molecules in the final polyaromatic polyamine mixture is greater than 25 wt % but less than about 50 wt % of the total polyaromatic polyamine and where the monoaromatic diamine is used in an amount between about 5 and about 30 mole %, preferably between about 10 and about 25 mole %, relative to 100 mole % of the monoaromatic monoamine used for the production and where the polyamine manufacturing process is carried out by economically beneficial means especially by using relatively low levels of acid catalyst and without operating at economically unattractive slow rates. The polyaromatic polyamines can be used as such or, preferably, may be further reacted to produce the corresponding polyaromatic polyisocyanates. The polyaromatic polyisocyanates can be used as such or can be blended or reacted further with other isocyanate-containing products or with isocyanate-reactive products.

### DETAILED DESCRIPTION OF THE INVENTION

Production of polyamine mixtures using formaldehyde and two or more aromatic amines leads to a complex mixture of compounds. The following description based on the use of aniline and 2,4-TDA, with aniline as the main aromatic amine and 2,4-TDA as a relatively minor component, is provided for clarity but it is to be understood not to limit the scope of the invention.

Products formed when producing polyamine mixtures from formaldehyde, aniline and 2,4-TDA include the dinuclear species AA & AB, the trinuclear species AAB & ABA, the tetranuclear species AAAB & AABA etc. in various isomeric forms [where A denotes aniline and B denotes 2,4-TDA]. Since here B is a minor component, the species BB, ABB, AABB, etc would normally only be present at lower levels and are not considered in the following descriptions, even though their presence in the mixtures might affect the properties of the polyamines, the derived corresponding polyisocyanates or the derived reaction products of the polyisocyanates with various other products. Preparations where the amount of TDA is greater than about 30 mole % relative to 100 mole % of aniline used have significant quantities of BB, ABB, AABB, etc and are not desired, especially as these types of components adversely affect the overall viscosities of the final polyamine and derived polyisocyanate mixtures. Preparations where the amount of TDA is less than about 5 mole % relative to 100 mole % of aniline used have properties that are not significantly different from preparations made without TDA.

In the following description, the following terms are used:

MDA: methylene dianiline (also known as diaminodiphenylmethane) which, unless specified otherwise, also denotes the various isomeric forms and their mixtures.
PMDA: the complex mixture of polyaromatic polyamines, including MDA, formed by the acid-catalysed reaction of aniline with formaldehyde.
TDA: toluene diamine (also known as diaminotoluene) which, unless specified otherwise, also denotes the various isomeric forms and their mixtures.
MTA: methylene di-phenylene, mono-methyl, triamine (also known as triamino-methyl-diphenylmethane) which, unless specified otherwise, also denotes the various isomeric forms and their mixtures.
PMTA: the complex mixture of polyaromatic polyamines, including MDA and MTA, formed by the acid-catalysed reaction of aniline and TDA with formaldehyde.

Conversion of the polyamines to the corresponding polyisocyanates by phosgenation produces respectively MDI, PMDI, TDI, MTI and PMTI.

Thus, present in the polyamine mixture will be:

4,4′-MDA (plus the 2,4′-MDA and 2,2′-MDA isomers—not shown)

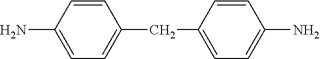

and various isomers of MTA

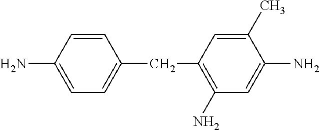

and isomers of the different types of tri-nuclear polyamines

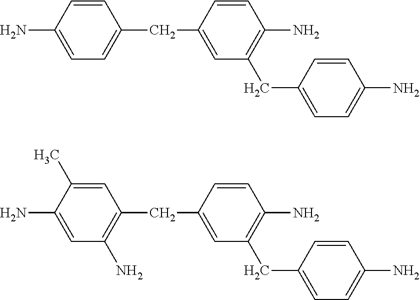

and isomers of the different types of tetra-nuclear and higher molecular weight polyamines (not shown).

It is to be understood that these structures are only representative of some of the compounds present in the polyamine mixture. There will also be higher molecular weight oligomers and various isomers of each of the different molecular weight oligomers. Various impurity species for example molecules with N—CH₃ [N-methyl] groups will also be present with a range of oligomeric and isomeric forms.

Phosgenation of the polyamine mixture gives the corresponding polyisocyanates and relatively minor levels of side products produced from various reactions between species (including but not limited to ureas, biurets, uretonimines, dimers, trimers, etc) and products derived from impurities.

Thus, present in the polyisocyanate mixture will be:

4,4′-MDI (plus the 2,4′-MDI and 2,2′-MDI isomers—not shown)

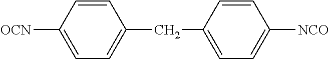

and various isomers of MTI

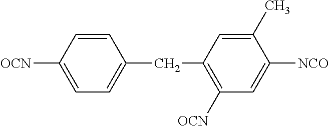

and isomers of the different types of tri-nuclear polyisocyanates

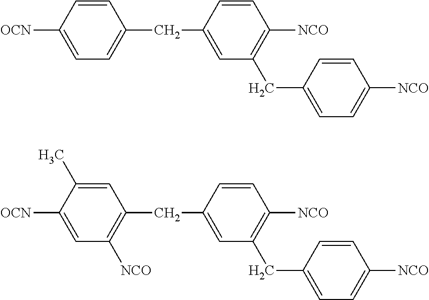

and isomers of the different types of tetra-nuclear and higher molecular weight polyisocyanates (not shown).

It is to be understood that these structures are only representative of some of the compounds present in the polyisocyanate mixture. There will also be higher molecular weight oligomers and various isomers of each of the different molecular weight oligomers and side products produced from various reactions between species. Various impurity species will also be present.

For simplicity, the entire mixture of all the isomers of all the AA, AB, AAB, ABA, AAAB, AABA, etc. polyamines is termed PMTA and the corresponding mixture of polyisocyanates is termed PMTI.

Instead of formaldehyde, materials capable of yielding the desired “CH₂O” species under the conditions of the process may also be used, for example, paraformaldehyde, dimethoxymethane (formal) and aqueous solutions of formaldehyde (formalin) at various concentrations. Stabilisers such as methanol, polyvinylalcohol, etc. may be present in the formalin. Gaseous formaldehyde may also be employed [WO 2007/065767]. Preferably formalin is used.

The acid catalyst used may be a mineral acid such as hydrochloric or sulphuric acid. Use of hydrochloric acid or gaseous hydrogen chloride in combination with aniline with an appropriate water content [WO 2007/065767] is preferred.

Examples of mononuclear aromatic monoamines which can be used include aniline, o- and m-substituted anilines such as toluidines and alkyl anilines, chloroanilines, anisidines and nitroanilines. Preferably aniline is used.

Examples of diamines or higher functionality amines which can be used include the diaminobenzene isomers such as 1,3-phenylene diamine, alkyl-substituted diaminobenzenes such as the isomers of TDA, isomers of methylene diphenylene diamine such as 4,4′-MDA and mixtures of various isomers and homologues of aniline-formaldehyde condensates known generally as PMDA. Particularly suitable mixtures are those containing from about 65 wt % to about 80 wt % 2,4-tolulene diamine and the balance 2,6-tolulene diamine. The commercially available mixture containing about 80 wt % 2,4- and about 20 wt % 2,6-tolulene diamine is very useful.

It is to be understood that any amine described within the context of the present invention refers to both the free amine and combined as an amine salt where appropriate. An additional benefit of the present invention is the production of polyisocyanate mixtures corresponding to the particular polyamine mixtures with improved reactivity due to the relatively low levels of so-called hydrolysable chlorine impurities, particularly from production of the polyamines with low levels of impurities especially those containing N-methyl groups. We have found that this is particularly accomplished by addition of the TDA after the reaction of formaldehyde with the acidified aniline.

Other additional benefits arising from various embodiments of the present invention include:

(a) the process can be operated as a batch, continuous or semi-continuous process;
(b) the reactants can be brought together by a variety of different methods such as dip-pipes into continuous stirred tank reactors or, preferably, by injection into one or more flowing streams through a number of injection points with mixing with static mixers or other mixing devices;
(c) the mono-amine and di-amine reactants may be provided in pure form or with low levels of impurities which do not affect the polyamine or polyisocyanate production or work-up processes nor have significant detrimental effects on final polyisocyanate product quality or derived polyurethane products;
(d) the formaldehyde may be provided in any suitable form, for example, as a gas, as solid paraformaldehyde or, preferably, as an aqueous solution (commonly termed formalin). The formalin may be of any suitable concentration, preferably greater that 40 wr % formaldehyde and may contain various stabilisers such as methanol and/or polyvinylalcohol. Preferably, the formalin contains low levels of di- and poly-valent metal ions to avoid formation of a rag layer after neutralisation of the acid reaction mixture;
(e) the hydrochloric acid may be provided as an aqueous solution, preferably with concentration greater that 30 wt % HCl or, optionally, it may be created in situ by absorption of gaseous hydrogen chloride, optionally derived from a phosgenation plant, either in to water or by absorption in to aniline containing an appropriate amount of water to avoid formation of aniline hydrochloride solids;
(f) preferably, the mono-amine may be mixed with the acid catalyst prior to introduction of the formaldehyde, the resulting reaction mixture then being reacted with the di-amine, optionally dissolved in additional mono-amine or solvent, and subsequently heated to higher temperatures to complete the rearrangement reactions. However, the acid addition may be split into aliquots. The final temperature of the reaction mixture is preferentially at least 120° C.;
(g) the process for the production and work-up of the polyamine mixture and the subsequent process for the production and work-up of the polyisocyanate can be monitored and optionally controlled by means of a range of on-line analytical techniques such as near-infrared spectroscopy, UV-visible spectroscopy, gas chromatography, liquid chromatography, nuclear magnetic resonance spectrometry, mass spectrometry and the like, optionally using chemometric or other numerical or computer-based techniques for data processing;
(h) the process for the production and work-up of the polyamine mixture may be retro-fitted to existing plant designed and constructed for the production of PMDA with relatively straightforward additional engineering design and construction. Likewise, conversion of the PMTA to PMTI can be carried out in existing PMDI production facilities with relatively straightforward additional engineering design and construction.

The process used in converting the polyamine mixture to the corresponding polyisocyanate mixture can be any of those described in the prior art or used commercially hitherto.

The object of the present invention in providing a process for the production of certain polyamine mixtures to be used as intermediates in the production of the corresponding polyisocyanates avoiding the drawbacks of the prior art will now be described in terms of the reactions occurring using formaldehyde (as an aqueous solution), aniline (as the mononuclear aromatic monoamine) and the so-called “80:20” mixture of 2,4′-TDA and 2,6′-TDA (as the mononuclear aromatic diamine—hereafter simply referred to as TDA) although it is to be understood that such a description is provided for the purpose of clarity and is not limiting in any way. Likewise, descriptions of the chemical reactions or chemical species present during the reactions are also given for the benefit of clarity only and not as any distinguishing aspect of the invention.

Aniline and hydrochloric acid are mixed together and, optionally, the temperature of the resulting mixture is controlled to a prescribed level, preferably in the range 30 to 90° C., preferentially in the range 40 to 65° C. Formaldehyde is added to the aniline/hydrochloric acid mixture, optionally by means of mixing equipment, whilst maintaining the temperature below a prescribed level, preferably in the range 30 to 90° C., preferentially in the range 40 to 65° C. This mixture may be maintained at low temperature for some time but, preferably, the TDA is added within a few minutes of the end of the formaldehyde addition at a temperature in the range 30 to 90° C., preferably in the range 45 to 85° C., still preferentially in the range 50 to 70° C. The preferred ranges described above give the most economically beneficial rate. The TDA may be added as a solid or preferentially in molten form or in solution in a solvent or, preferably, pre-dispersed in aniline. Normally the TDA is pre-dispersed in the minimum amount of aniline required to keep the TDA in solution as it is preferable for any given overall recipe to have the maximum amount of aniline present for the reaction with formaldehyde. Optionally, some of the hydrochloric acid may also be added at this time, either separately or included with the aniline/TDA.

Overall, the relative amounts of the various species brought together are:

HCl: in the range 0.05 to 0.4 moles per mole of formaldehyde, preferably 0.1 to 0.35 moles per mole of formaldehyde.
Total amine: in the range 2.6 to 3.1 moles per mole of formaldehyde, preferably 2.7 to 3.0 moles per mole of formaldehyde and where the TDA is between about 5 and about 30 mole %, preferably between about 10 and about 25 mole %, relative to 100 mole % of the aniline used for the production.

After addition of the TDA, the reaction mixture is brought to higher temperatures for an extended time, either following a simple linear temperature ramp or as a more complex temperature/time profile in order to convert the various secondary amine species [aminobenzylanilines—ABA's] to primary amines and thus obtain the desired mixture of polyamine oligomers and their corresponding isomer ratios. Preferably, the final temperature at this stage is greater than 100° C., preferably greater than 120° C., in order to carry out the final chemical rearrangements at an economically beneficial rate. Such temperatures require that the reaction mixture is maintained above atmospheric pressure. Determination of the desired end-point of the reaction can be determined by on-line analysis, by sampling followed by off-line analysis or by trial. The relationship between the overall ratios of the reactants viz: aniline/TDA/formaldehyde/HCl, the temperature/time profile and the exact final polyamine composition can be determined by trial.

Neutralisation of the acidic reaction mixture is then carried out by reaction with a suitable quantity of base. Preferably, this involves reaction with a slight stoichiometric excess of aqueous sodium hydroxide although any of the neutralisation options described in the prior art may also be applied. The neutralisation may be carried out on the polyamine mixture after cooling below the final reaction temperature or, preferably, at substantially the same temperature as the reaction in suitably designed equipment capable of withstanding the high temperatures and elevated pressures. Separation of the organic phase comprising of the predominantly aniline/TDA/polyamine mixture and the brine phase is then carried out with conventional phase separation devices. Secondary washing and subsequent separation of the phases is also preferentially carried out viz: the organic phase can be washed with additional aqueous material and the brine phase can be washed with additional aniline of suitable quality i.e. impurities may be present in the washing fluids.

Unreacted aniline and TDA are then removed from the organic mixture by any appropriate means preferentially by fractional distillation in one or more steps, to yield the final mixture of desired polyaromatic polyamines with amounts of the free mononuclear amines preferably below or substantially below a total of 200 ppm by weight, preferably below 50 ppm by weight. Water is also removed at this stage. The aniline and TDA may be recycled.

To ensure very low residual TDA going forward to phosgenation, the removal of the mononuclear amines is generally accompanied by removal of some higher molecular weight compounds such as, for example, dinuclear polyamines. The recycling stream of aniline, TDA and higher molecular weight polyamines could be subjected to further treatment, for example, fractional distillation, to modify the composition before addition back to the process. However, it is preferable to use the unmodified stream containing predominantly mononuclear amines such as aniline and TDA plus the higher molecular weight polyamines and add this stream back to the production process. The presence of the higher molecular weight compounds in the recycle will have a small effect on the composition of the polyamine mixture produced but this can be adequately compensated for by changes to the ratios of the various reactants viz: aniline, TDA, formaldehyde. The composition of the recycling stream can be monitored by on-line or off-line analysis for process control. Examples of suitable techniques include IR, Raman, NIR and UV-Vis spectroscopies or LC, HPLC, GC, GC-MS or NMR techniques, where these abbreviations have their well-known conventional meanings.

All of these so-called work-up process steps (neutralisation, phase separation, washing, distillation, etc) can be carried out according to any of the methods described or illustrated in the prior art or their combinations or generally according to such methods when applied with and in the context of modern process engineering methods and standards for example in relation to energy efficiency, energy integration, unit operation efficiency, etc. as is well known and understood to those skilled in the art.

After removal of unreacted aniline and TDA, the polyamine mixture may be used directly for example as the curing agent for epoxy resins or may be hydrogenated to form the corresponding mixture of cycloaliphatic polyamines or may be processed further, for example by fractional distillation or fractional crystallisation, to produce more than one stream of different polyamine composition for example for production of the corresponding polyisocyanates.

After removal of unreacted aniline and TDA, the polyaromatic polyamine mixture can be converted to the corresponding polyisocyanates by reaction with phosgene in any of the methods described in the prior art.

The phosgenation reaction can be carried out by any of the many and well known variations described in the prior art.

For example, the PMTA can be dissolved in chlorobenzene to a level of typically 10 to 50 wt %, preferably 20 to 35 wt %, the resulting solution then being introduced into reaction vessels typically by means of special mixing devices such as jet nozzles or high shear mixtures by means of which the amine blend is thoroughly and intimately mixed with phosgene, also optionally in solution, preferably in the same solvent as the PMTA. Reaction temperature at this stage is typically in the range 50 to 150° C., preferably 75 to 95° C. The product of this initial reaction stage may be worked up immediately or there may be additional reaction, optionally in additional reaction vessels, optionally including addition of phosgene, for further digestion of reaction intermediates and/or by-products. Many pressure and temperature regime variations are known from the prior art and many variations in process equipment can be employed.

On completion of the phosgenation reaction, the crude PMTI product can be separated from excess phosgene, product HCl, and reaction solvent by any means known to those skilled in the art, typically by distillation, and subjected to further work up such as the well established thermal cracking of impurity compounds known as “dechlorination”. The mixture of MDI di-isocyanate isomers, MTI isomers and all the various PMTI homologues in their various isomeric forms can be used as such or further refined to give various MDI di-isocyanate products or predominantly MTI products or MDI/MTI mixtures or polymeric mixtures, typically by fractional distillation or fractional crystallisation. All these process steps can be carried out in batch, continuous or semi-continuous modes.

The derived polyisocyanate compositions are thus available for further use for a wide variety of applications, including use without further modification as the isocyanate component of polyurethanes or other applications, as the isocyanate component of prepolymers (by reaction with, for example, di- or polyfunctional polyether or polyester polyols or other materials with isocyanate-reactive functional groups) or variants (by formation of uretonimine, isocyanurate or other functional groups) and for further processing.

This further processing such as by means of fractional distillation and/or fractional crystallisation can be used to separate some or all of the lower molecular weight components in the polyisocyanate mixture to produce a polyisocyanate mixture with a different composition. The separated lower molecular weight polyisocyanates may be MDI or MTI or mixtures thereof and are understood to encompass all possible separate pure or substantially pure individual isomers of specific mixtures. Thus, this further processing such as by means of further fractional distillation and/or further fractional crystallisation can be used to produce other compositions such as, for example, relatively pure 4,4′-MDI; mixtures of predominantly 4,4′-MDI and 2,4′-MDI; mixtures of MDI and MTI isomers in various proportions; mixtures of predominantly MTI isomers; or relatively pure individual MTI isomers.

The separated lower molecular weight polyisocyanates may themselves be used with or without further modification as the isocyanate component of polyurethanes or in other applications, as the isocyanate component of prepolymers (by reaction with, for example, di- or polyfunctional polyether or polyester polyols or other materials with isocyanate-reactive functional groups) or variants (by formation of uretonimine, isocyanurate or other functional groups) and for further processing.

### EXAMPLES

The present invention is explained by means of the following examples, but the present invention is not limited to these examples.

### Comparative Example 1

Toluene diamine (the “80/20” mixture of 2,4- and 2,6-TDA isomers) was preheated in an oven at 95° C.

In a beaker, 75.4 g (0.81 mol.) aniline [purity 99.5%] was weighed, then heated on a stirrer/hotplate and 75.0 g (0.61 mol.) of the liquid TDA was added, mixed, transferred to a sample bottle and stored in an oven at 65° C.

In a nitrogen current, 466.0 g (4.98 mol.) of aniline was added to a 1-liter pressure-reactor, agitation was started and the aniline was temperature conditioned at 20° C. Then 152.8 g (1.27 mol.) of 30.4% hydrochloric acid was added drop-wise in 14 minutes, such that the temperature increased up to 41° C. The solution was conditioned for 10 minutes at 40° C.

Next, 146.8 g (2.30 mol.) of 47% formaldehyde was pumped in to the stirred reactor during 180 minutes with a flow rate of 0.71 ml/min while maintaining the temperature at 40° C.

Next the temperature was increased and after reaching 60° C., 141.1 g of the toluene diamine/aniline solution (respectively 0.58 and 0.76 moles) was added. The overall ratio of reactants used at this stage is thus An/TDA/F/HCl=2.50/0.25/1.0/0.55.

The reactor was closed, the temperature was increased to 90° C. and the reactor was pressurized up to 0.5 bar. The temperature was maintained for 30 minutes at 90° C., followed by a temperature increase to 130° C. in 20 minutes, and maintaining the temperature for 90 minutes at 130° C.

Next the temperature was decreased in 15 minutes to 80° C. and the solution collected and stored for convenience in an oven at 80° C.

748 g of the reaction mixture was transferred to a 1-liter reactor and heated to 100° C. 103 g (1.29 mol) of sodium hydroxide solution (50 wt %) was added and an additional amount of 148 g hot water also added, stirred for 5 minutes: separation of the layers was easily achieved in 5 minutes. The bottom layer (water phase) was discharged and 96 g. hot water was added to the remaining organic layer, stirred for 5 minutes and the phases allowed to separate for 5 minutes. The bottom layer (organic phase) was collected and the top layer (water phase) was discharged. The organic phase was rinsed for another 4 times with 100 g of hot water each time to yield 691 g of the raw amine mixture.

The composition of the raw amine as determined by gel permeation chromatography using refractive index detection was as follows:

| | |
|---|---|
| Aniline | 28.1 wt % |
| Toluene diamine isomers | 2.1 wt % |
| Diphenylmethanes | 43.8 wt % |
| Triphenylmethanes | 16.5 wt % |
| Polyamines (tetra + penta + etc) | 9.4 wt % |

Thus, the amount of di-nuclear species in the polyaromatic polyamine mixture is about 63%.

The diphenyl methanes are a mixture of MDA isomers and MTA isomers. The individual MDA isomers can be determined by gas chromatographic analysis using flame ionisation detection which gave the following results:—

| | |
|---|---|
| 4,4′-MDA | 30.2 wt % |
| 2,4′-MDA | 3.8 wt % |
| 2,2′-MDA | 0.1 wt % |

The amount of MTA isomers can be estimated by difference as 9.7 wt % [43.89−30.2−3.8−0.1]. The presence of minor amounts of diphenylmethane impurity species such as N-methylated variations of the main compounds [e.g. H₂N-Ph-CH₂-Ph-NH—CH₃] are acknowledged to give some inaccuracies in the determination of the MTA concentration, but these will not detract significantly from the calculated abundance.

After removal of the bulk of the unreacted mononuclear amines by distillation, the total amount of N-methyl groups present in the polyamine mixture was measured by ¹H-NMR of a solution of the polyamine in deuterated chlorobenzene, after removal of labile H-atoms by exchange with D₂O. The ratio of N-methyl groups compared to methylene groups between aromatic rings was found to be 0.2 to 99.8 (no unreacted amino-benzyl-aniline species were detected: —CH₂—N— peaks not observed).

### Comparative Example 2

Toluene diamine (the “80/20” mixture of 2,4- and 2,6-TDA isomers) was preheated in an oven at 95° C. In a beaker, 75.4 g (0.81 mol.) aniline was weighed, heated on stirrer/hotplate and 150.2 g (1.23 mol.) of liquid TDA was added, mixed, transferred to a sample bottle and stored in an oven at 65° C.

In a nitrogen current, 466.3 g (4.98 mol.) of aniline [purity 99.5%] was added to a 1-liter pressure-reactor, agitation started and conditioned at 20° C., then 152.8 g (1.27 mol.) of 31.3% hydrochloric acid was added drop wise in 8 minutes; the temperature increased to 39° C. The solution was conditioned for 10 minutes at 40° C.

Next, 146.8 g (2.30 mol.) of 47% formaldehyde was pumped into the stirred reactor during 180 minutes with a flow rate of 0.71 ml/min while maintaining the temperature at 40° C.

Next the temperature was increased to 60° C. when 214.7 g of the toluene diamine/aniline solution (respectively 1.17 and 0.77 moles) was added. The overall ratio of reactants used at this stage is thus An/TDA/F/HCl=2.50/0.5/1.0/0.57.

The reactor was closed, the temperature was increased to 90° C. and the reactor was pressurized up to 0.5 bars. The temperature was maintained for 30 minutes at 90° C., followed by a temperature increase to 130° C. in 20 minutes (pressure to 1.5 barg), and maintaining the temperature for 90 minutes at 130° C.

Next the temperature was decreased in 15 minutes to 80° C. and the solution collected and stored for convenience in an oven at 80° C.

805 g of the reaction mixture was neutralized and worked-up in the same way as in the previous example to yield 798 g raw amine.

The composition of the raw amine as determined by gel permeation chromatography using refractive index detection was as follows:

| | |
|---|---|
| Aniline | 30.9 wt % |
| Toluene diamine isomers | 2.4 wt % |
| Diphenylmethanes | 41.5 wt % |
| Triphenylmethanes | 17.3 wt % |
| Polyamines (tetra + penta + etc) | 7.8 wt %: |

Thus, the amount of di-nuclear species in the polyaromatic polyamine mixture is about 62%.

The diphenyl methanes are a mixture of MDA isomers and MTA isomers. The individual MDA isomers can be determined by gas chromatographic analysis using flame ionisation detection which gave the following results:

| | |
|---|---|
| 4,4′-MDA | 16.5 wt % |
| 2,4′-MDA | 2.4 wt % |
| 2,2′-MDA | 0.1 wt % |

The amount of MTA isomers can be estimated by difference as 22.5 wt % [41.59−16.5−2.4−0.1]. The presence of minor amounts of diphenylmethane impurity species such as N-methylated variations of the main compounds [e.g. H₂N-Ph-CH₂-Ph-NH—CH₃] are acknowledged to give some inaccuracies in the determination of the MTA concentration, but these will not detract significantly from the calculated abundance.

The ratio of N-methyl groups compared to methylene groups between aromatic rings was found to be 0.46 to 99.54 (no unreacted amino-benzyl-aniline species were detected: —CH₂—N— peaks not observed).

### Example 3

Toluene diamine (the “80/20” mixture of 2,4- and 2,6-TDA isomers) was preheated in an oven at 95° C.

In a beaker, 75.4 g (0.81 mol.) aniline [purity 99.5%] was weighed, then heated on a stirrer/hotplate and 75.2 g (0.62 mol.) of the liquid TDA was added, mixed, transferred to a sample bottle and stored in an oven at 65° C.

In a nitrogen current, 468.0 g (5.0 mol.) of aniline was added to a 1-liter pressure-reactor, agitation was started and the aniline was temperature conditioned at 20° C. Then 25 g (0.21 mol.) of 31.3% hydrochloric acid was added drop-wise in 1.5 minutes, such that the temperature increased up to 26° C. The solution was conditioned for 10 minutes at 40° C.

Next, 146.8 g (2.30 mol.) of 47% formaldehyde was pumped in to the stirred reactor during 178 minutes with a flow rate of 0.71 ml/min while maintaining the temperature at 40° C.

Next the temperature was increased and after reaching 60° C., 144 g of the toluene diamine/aniline solution (respectively 0.59 and 0.77 moles) was added. The overall ratio of reactants used at this stage is thus An/TDA/F/HCl=2.50/0.26/1.0/0.09.

The reactor was closed, the temperature was increased to 90° C. and the reactor was pressurized up to 0.5 bar. The temperature was maintained for 30 minutes at 90° C., followed by a temperature increase to 130° C. in 20 minutes, and maintaining the temperature for 90 minutes at 130° C.

Next the temperature was decreased in 15 minutes to 80° C. and the solution collected and stored for convenience in an oven at 80° C.

634 g of the reaction mixture was transferred to a 1-liter reactor and heated to 100° C. 18 g (0.23 mol) of sodium hydroxide solution (50 wt %) was added and the mixture stirred for 5 minutes: separation of the layers was easily achieved in 5 minutes.

The bottom layer (organic phase) was collected and the top layer (aqueous phase) was discharged.

The organic phase was returned to the reactor and 122 g. hot water was added, stirred for 5 minutes and the phases allowed to separate for 5 minutes. The bottom layer (organic phase) was collected and the top layer (water phase) was discharged. The organic phase was rinsed for another 4 times with 100 g of hot water each time to yield 544 g of the raw amine mixture.

The composition of the raw amine as determined by gel permeation chromatography using refractive index detection was as follows:

| | |
|---|---|
| Aniline | 30.8 wt % |
| Toluene diamine isomers | 0.7 wt % |
| Diphenylmethanes | 35.2 wt % |
| Triphenylmethanes | 20.3 wt % |
| Polyamines (tetra + penta + etc) | 13.1 wt %: |

Thus, the amount of di-nuclear species in the polyaromatic polyamine mixture is about 51%.

The diphenyl methanes are a mixture of MDA isomers and MTA isomers. The individual MDA isomers can be determined by gas chromatographic analysis using flame ionisation detection which gave the following results:

| | |
|---|---|
| 4,4′-MDA | 21.0 wt % |
| 2,4′-MDA | 4.1 wt % |
| 2,2′-MDA | 0.3 wt % |

The amount of MTA isomers can be estimated by difference as 9.8 wt % [35.29−21.0−4.1−0.3]. The presence of minor amounts of diphenylmethane impurity species such as N-methylated variations of the main compounds [e.g. H₂N-Ph-CH₂-Ph-NH—CH₃] are acknowledged to give some inaccuracies in the determination of the MTA concentration, but these will not detract significantly from the calculated abundance.

After removal of the bulk of the unreacted mononuclear amines by distillation, the total amount of N-methyl groups present in the polyamine mixture was measured by ¹H-NMR of a solution of the polyamine in deuterated chlorobenzene, after removal of labile H-atoms by exchange with D₂O. The ratio of N-methyl groups compared to methylene groups between aromatic rings was found to be 0.1 to 99.9 (no unreacted amino-benzyl-aniline species were detected: —CH₂—N— peaks not observed).

### Example 4

Firstly, 100.11 g of aniline (1.075 mol) [purity 100%] was placed in a 3 necked flask fitted with a reflux condenser (cold water) and a dropping funnel, then 7.39 g (0.075 mol) of 37% hydrochloric acid was added slowly under stirring and the temperature was monitored and increased to 40° C. (eventually heated).

Separately, 31.17 g (0.25 mol) of toluene diamine (the “80/20” mixture of 2,4- and 2,6-TDA isomers, 98% purity) was placed in a separated flask and mixed with 16.30 g (0.175 mol) of aniline under stirring at 60° C. (reflux) for 30 minutes.

When the aniline/HCl mixture was at 40° C., 40.58 g (0.5 mol) of 37% formaldehyde solution, placed in the dropping funnel, was added dropwise at a constant rate under mixing over a period of 2 hours maintaining the temperature at about 40° C.

Five minutes after the end of formaldehyde addition, the temperature was increased to 60° C. over 20 minutes after which the TDA/aniline mixture (0.25 mol TDA/0.175 mol aniline) was then added. The mixture was maintained at 60° C. for ca. 1 hour. The overall ratio of reactants used at this stage is thus An/TDA/F/HCl=2.50/0.5/1.0/0.15.

Next the temperature was increased to 90° C. over 20 minutes and kept at this temperature for 16 h (over night).

The temperature was decreased to 50° C. in 15 minutes. Then 7.2 g (0.09 mol) of sodium hydroxide solution (50 wt %) was added slowly for the neutralization. The mixture was kept under stirring for 30 minutes. After cooling down to ambient temperature, the solution was transferred to a separating funnel for phase separation.

The bottom layer (water phase) was discharged and the organic phase analyzed.

The composition of the raw amine as determined by gel permeation chromatography using refractive index detection was as follows:

| | |
|---|---|
| Aniline | 35.48 wt % |
| Toluene diamine isomers | 0.27 wt % |
| Diphenylmethanes | 21.71 wt % |
| Triphenylmethanes | 31.83 wt % |
| Polyamines (tetra + penta + etc) | 10.68 wt % |

Thus, the amount of di-nuclear species in the polyaromatic polyamine mixture is about 34%.

The diphenyl methanes are a mixture of MDA isomers and MTA isomers. The individual MDA isomers can be determined by gas chromatographic analysis using flame ionisation detection which gave the following results:

| | |
|---|---|
| 4,4′-MDA | 6.74 wt % |
| 2,4′-MDA | 1.7 wt % |
| 2,2′-MDA | 0.15 wt % |

The amount of MTA isomers can be estimated by difference as 13.12 wt % [21.719−6.74−1.7−0.15]. The presence of minor amounts of diphenylmethane impurity species such as N-methylated variations of the main compounds [e.g. H₂N-Ph-CH₂-Ph-NH—CH₃] are acknowledged to give some inaccuracies in the determination of the MTA concentration, but these will not detract significantly from the calculated abundance.

The total amount of N-methyl groups present in the polyamine mixture was measured by ¹H-NMR of a solution of the polyamine in deuterated chlorobenzene, after removal of labile H-atoms by exchange with D₂O. The ratio of N-methyl groups compared to methylene groups between aromatic rings was found to be 0.34 to 99.66 (no unreacted amino-benzyl-aniline species were detected: —CH₂—N— peaks not observed).

Hot water was added to the remaining organic layer, stirred for 30 minutes and the phases allowed to separate for 30 minutes. The bottom layer (organic phase) was collected and the top layer (water phase) was discharged. The organic phase was rinsed for another 5 times with 75 g of hot water each time to obtain the raw amine mixture.

### Example 5

95.5 g of aniline (1.025 mol) [purity 100%] was placed in a 3 necked flask fitted with a reflux condenser (cold water) and a dropping funnel, then 7.39 g (0.075 mol) of 37% hydrochloric acid was added under stirring and the temperature was monitored and increased to 40° C. (eventually heated).

Separately, 36.65 g (0.6 mol) of toluene diamine (the “80/20” mixture of 2,4- and 2,6-TDA isomers, 98% purity) was placed in a separated flask and mixed with 16.30 g (0.175 mol) of aniline under stirring at 60° C. (reflux) for 30 minutes.

When the aniline/HCl mixture was at 40° C., 40.58 g (0.5 mol) of 37% formaldehyde solution, placed in the dropping funnel, was added dropwise at a constant rate over a period of 2 hours maintaining the temperature at about 40° C.

Five minutes after the end of formaldehyde addition, the temperature was increased to 60° C. over 20 minutes after which the TDA/aniline mixture (0.6 mol TDA/0.175 mol aniline) was added, under high mixing, in 3 equal parts separated equally over the course of 1 hour. The reaction mixture was kept at 60° C. for 1 hour.

The overall ratio of reactants used at this stage is thus An/TDA/F/HCl=2.4/0.6/1.0/0.15.

The temperature was then increased to 90° C. over 20 minutes and kept at this temperature for 16 h (over night).

The temperature was decreased to 50° C. over 15 minutes. Then 7.2 g (0.09 mol) sodium hydroxide solution (50 wt %) was added slowly for the neutralization. The mixture was kept under stirring for 30 minutes. After cooling down to ambient temperature, the solution was transferred to a separating funnel for phase separation. The bottom layer (water phase) was discharged and the organic phase analyzed.

The composition of the raw amine as determined by gel permeation chromatography using refractive index detection was as follows:

| | |
|---|---|
| Aniline | 32.71 wt % |
| Toluene diamine isomers | 0.17 wt % |
| Diphenylmethanes | 22.52 wt % |
| Triphenylmethanes | 32.19 wt % |
| Polyamines (tetra + penta + etc) | 12.22 wt % |

Thus, the amount of di-nuclear species in the polyaromatic polyamine mixture is about 34%.

The diphenyl methanes are a mixture of MDA isomers and MTA isomers. The individual MDA isomers can be determined by gas chromatographic analysis using flame ionisation detection which gave the following results:

| | |
|---|---|
| 4,4′-MDA | 5.66 wt % |
| 2,4′-MDA | 1.287 wt % |
| 2,2′-MDA | 0.12 wt % |

The amount of MTA isomers can be estimated by difference as 15.46 wt % [22.529−5.66−1.287−0.12]. The presence of minor amounts of diphenylmethane impurity species such as N-methylated variations of the main compounds [e.g. H₂N-Ph-CH₂-Ph-NH—CH₃] are acknowledged to give some inaccuracies in the determination of the MTA concentration, but these will not detract significantly from the calculated abundance.

The total amount of N-methyl groups present in the polyamine mixture was measured by ¹H-NMR of a solution of the polyamine in deuterated chlorobenzene, after removal of labile H-atoms by exchange with D₂O. The ratio of N-methyl groups compared to methylene groups between aromatic rings was found to be 0.29 to 99.71 (no unreacted amino-benzyl-aniline species were detected: —CH₂—N— peaks not observed).

Hot water was added to the remaining organic layer, stirred for 30 minutes and the phases allowed to separate for 30 minutes. The bottom layer (organic phase) was collected and the top layer (water phase) was discharged. The organic phase was rinsed for another 5 times with 75 g of hot water each time to obtain the raw amine mixture.

### Example 6

Toluene diamine (the “80/20” mixture of 2,4- and 2,6-TDA isomers) was preheated in an oven at 95° C. In a beaker, 75.4 g (0.81 mol.) aniline was weighed, heated on stirrer/hotplate and 150.2 g (1.23 mol.) of liquid TDA was added, mixed, transferred to a sample bottle and stored in an oven at 65° C.

In a nitrogen current, 466.3 g (4.98 mol.) of aniline [purity 99.5%] was added to a 1-liter pressure-reactor, the agitation was started. The contents were conditioned at 20° C., then 93.8 g (0.8 mol.) of 31.3% hydrochloric acid was added dropwise over 8 minutes; the temperature increased to 39° C. The solution was conditioned for 10 minutes at 40° C.

Next, 146.8 g (2.30 mol.) of 47% aqueous formaldehyde was pumped in to the stirred reactor over 120 minutes with a flow rate of 0.71 ml/min while maintaining the temperature at 40° C.

The reactor temperature was then set to increase to 90° C. over 30 minutes. When the reactor contents reached 60° C., 214.7 g of the TDA/aniline solution (respectively 1.17 and 0.77 moles) were added. The overall ratio of reactants used at this stage is thus An/TDA/F/HCl=2.50/0.5/1.0/0.35.

When the temperature reached 90° C., the reactor was pressurized up to 0.5 barg. The temperature was maintained for 60 minutes at 90° C., followed by a temperature increase to 120° C. over 20 minutes (pressure to 1.5 barg). This temperature was maintained for a further 120 minutes.

The temperature was then decreased to 80° C. over 15 minutes and the solution collected and stored for convenience in an oven at 80° C.

The reaction mixture was neutralized and worked-up in the same way as in Example 1. The aniline-water azeotrope present in the washed organic phase was distilled off with a rotavap by heating from 60 to 135° C. and under vacuum [pressure was adjusted in steps from −0.5 to −1.0 barg] thus ensuring the organic phase is free of excess aniline. These conditions were not sufficient to remove unreacted TDA completely.

The composition of the polyaromatic polyamine mixture after removal of the bulk of the unreacted mononuclear amines by distillation was determined by gel permeation chromatography using refractive index detection and was as follows:

| | |
|---|---|
| Diphenylmethanes | 48.7 wt % |
| Triphenylmethanes | 36.7 wt % |
| Polyamines (tetra + penta + etc) | 14.6 wt %: |

Thus, the amount of di-nuclear species in the polyaromatic polyamine mixture is about 49%.

The diphenyl methanes are a mixture of MDA isomers and MTA isomers. The individual MDA isomers can be determined by gas chromatographic analysis using flame ionisation detection which gave the following results:—

| | |
|---|---|
| 4,4′-MDA | 20.2 wt % |
| 2,4′-MDA | 3.5 wt % |
| 2,2′-MDA | 0 wt % |

The amount of MTA isomers can be estimated by difference as 25.0 wt % [48.7−20.2−3.5]. The presence of minor amounts of diphenylmethane impurity species such as N-methylated variations of the main compounds [e.g. H₂N-Ph-CH₂-Ph-NH—CH₃] are acknowledged to give some inaccuracies in the determination of the MTA concentration, but these will not detract significantly from the calculated abundance.

After removal of the bulk of the unreacted mononuclear amines by distillation, the total amount of N-methyl groups present in the polyamine mixture was measured by ¹H-NMR of a solution of the polyamine in deuterated chlorobenzene, after removal of labile H-atoms by exchange with D₂O. The ratio of N-methyl groups compared to methylene groups between aromatic rings was found to be 0.13 to 99.87 (no unreacted amino-benzyl-aniline species were detected: —CH₂—N— peaks not observed).

### Example 7

The PMTA obtained from Example 8 was converted to the corresponding isocyanate, PMTI, by phosgenation in monochlorobenzene (MCB). The phosgenation of the PMTA was carried out in a batch reactor following this procedure:

All the MCB to be used was pre-dried.

A 5% solution of the PMTA in MCB was prepared and this was filtered at 40° C. before being used.

Approximately 500 ml of MCB were added to the reactor and cooled to 10° C. Next phosgene was added to the reactor in a four-fold molar excess compared to the amine using a condenser with dry ice to keep the phosgene in the reactor. The mixture was cooled down to 10° C. again.

While mixing well, the amine solution was added to the reactor at such a rate that the temperature stayed below 40° C. A large quantity of solid intermediate products was formed.

The temperature was increased to about 110° C. over 1 hour until a clear solution was obtained.

Then the residual phosgene, HCl and some of the MCB was removed using a Wiped Film Evaporator operating at 150° C. and atmospheric pressure. The remainder of the MCB was removed using batch distillation with a nitrogen purge at 150° C. and 250 mbar.

The mixture was then kept for 20 minutes at 190° C. (250 mbar) under nitrogen and a further 10 minutes at 200° C. at atmospheric pressure to breakdown Cl-impurities (so-called dechlorination). The mixture was then passed through a Wiped Film Evaporator set-up [operating at 200° C. and 75 mbar with a 400 ml/min nitrogen flow] followed by rapid cooling to prevent the formation of excessive dimer by means of

The PMTI obtained had an isocyanate content [NCO value] of 35.52%.

1. A process for the preparation of polyaromatic polyamines comprising the step of reacting formaldehyde or related CH₂O species generating compounds with at least one monoaromatic monoamine and at least one monoaromatic compound containing at least two amino functions in the presence of an acidic catalyst where
a) the total amount of di-aromatic compounds in the polyaromatic polyamine mixture is in the range from about 25 wt % to about 50 wt % and
b) the amount of monoaromatic compound containing at least two amine functions is in the range 5 to 30 mole % relative to 100 mole % of the total amount of monoaromatic monoamines and
c) the amount of acidic catalyst used in the preparation of the polyaromatic polyamine mixture is less than about 0.4 moles per mole of formaldehyde or formaldehyde equivalents.

2. Process according to claim 1 wherein the monoaromatic monoamine comprises aniline.

3. Process according to claim 1 or 2 wherein the monoaromatic compound containing at least two amino functions comprises one or more of the isomers of toluene diamine or one or more isomers of diaminobenzene or their mixtures.

4. Process according to any one of the preceding claims wherein the amount of monoaromatic compound containing at least two amine functions is in the range 10 to 25 mole % relative to 100 mole % of the total amount of monoaromatic monoamines.

5. Process according to any one of the preceding claims wherein the acidic catalyst comprises hydrochloric acid.

6. Process according to claim 5 wherein the amount of hydrochloric acid is in the range 0.05 to 0.4 moles per mole of formaldehyde.

7. Process according to any one of the preceding claims wherein the total amount of amine compounds is in the range 2.6 to 3.1 moles per mole of formaldehyde.

8. Process according to any one of the preceding claims wherein in a first step the monoaromatic monoamine is reacted with formaldehyde in the presence of acidic catalyst and in a subsequent step the monoaromatic compound containing at least two amine functions is added.

9. Polyaromatic polyamines obtainable by the process as defined in any one of the preceding claims.

10. A process for the preparation of polyaromatic polyisocyanates comprising the step of phosgenating the polyaromatic polyamines as defined in claim 9.

11. Polyaromatic polyisocyanates obtainable by the process as defined in claim 10.
